(19) United States
(12) Patent Application Publication
(10) Pub. No.: US 2010/0089887 A1
(43) Pub. Date: Apr. 15, 2010

(54) Method for Controlling a Protection Visor

(76) Inventors: Helmut Friedl, Sipbachzell (AT); Uwe Kroiss, Wilhering (AT); Bank Sardy, Linz/Donau (AT)

Correspondence Address: COLLARD & ROE, P.C., 1077 NORTHERN BOULEVARD, ROSLYN, NY, 11576, US

(21) Appl. No.: 11/989,972

(22) PCT Filed: Aug. 3, 2006
(86) PCT No.: PCT/AT2006/000327

§ 371 Date: Feb. 4, 2008

(30) Foreign Application Priority Data
Aug. 30, 2005 (AT) A 1420/2005

(51) Int. Cl. B23K 9/10; A61F 9/06

(52) U.S. Cl. 219/130.21; 2/8.2

(57) ABSTRACT

The invention describes a method for controlling a protective shield (29) by means of a welding device (1), wherein a signal is transmitted to a welding helmet (28) by the welding device (1), whereupon an electrically activatable protective shield (29) is activated by a transmitting/receiving apparatus (31) integrated in the welding helmet (28), and the protective shield (29) is darkened. Here, during the welding process, i.e. after ignition of the electric arc (15), the protective shield (29) is activated by a control device (4) of the welding device (1) via the transmitting/receiving apparatuses (30, 31), wherein intensity and/or degree of darkening of the protective shield (29) is changed during the welding process by repetitively darkening and brightening the protective shield (29) in an alternating manner. Thus, a better welding-process observation is achieved for the welder.

The present invention relates to a method for controlling a protective shield, e.g. on a welding helmet, of a welding device, wherein a signal is transferred by the welding device to a transmitting/receiving apparatus, whereupon the electronically activatable protective shield wil be activated by a transmitting/receiving apparatus associated to the protective shield, e.g. integrated in the welding helmet, and the protective shield will be darkened.

From EP 1 202 832 B1 a control process for a welding plant with a welding device and a welding helmet for a user is known, wherein a starting signal to be sent to a control means of the welding device is generated for activating the welding process by actuating a starting switch provided on a welding torch or a welding device. When activating the starting switch a starting signal is transmitted to the welding helmet via a transmitting/receving apparatus, whereupon an electrically controlable protecting shield will be activated by the welding helmet by applying energy and, subsequently, the welding process, in particular ignition of the electric arc, will be started. This has the disadvantage that an activation of the protective shield is (here) effected only prior to or after the welding process so that the protective shield of the welding helmet is kept at almost the same darkness during the whole welding process.

U.S. Pat. No. 4,638,146 A shows a method and a device for protection of the eyes of a welder against welding light in electric-arc welding, wherein the magnetic field around the welding power supply cable is used as a triggering element for ensuring a darkening of the welding shield in time.

U.S. Pat. No. 5,208,688 A describes a welding helmet with a specific type of light filtering for ensuring an optimum protection of the welder's eyes.

U.S. Pat. No. 3,873,804 A, U.S. Pat. No. 6,067,129 A, U.S. Pat. No. 4,418,267 A and US 2005/0001155 A1 show different embodiments of a welding helmet and/or a safety device for welders, which, for obtaining optimum protection, can be controlled such that they darken the shield device at the point of time when the electric arc is ignited at the latest. There is no control of darkening of the welding shield during the welding process in none of the prior-art methods and devices.

The object of the invention resides in providing a method for controlling a welding helmet of a welding device in order to achieve better welding-process observation for the welder.

The object of the invention is achieved in that during the welding process, i.e. after ignition of the electric arc, the protective shield is activated by a control device of the welding device via the transmitting/receiving apparatus, wherein intensity and/or degree of darkening of the protective shield is changed during the welding process by repetitively darkening and brightening the protective shield in an alternating manner.

In doing so, it is advantageous that, by intentionally brightening the protective shield during the welding process, the welder or user can optimally observe the melting bath. Thus, the user can optimally adjust the welding device in case of bad welding results, since the user can exactly observe the different process states during a test welding. Thereby it is also achieved that the welder can see the surroundings, such as gap width, position of the parts, etc., better, thus being capable of guiding the welding torch optimally. By brightening the welding shield, the user is also provided with a very good view on the just produced weld, thus being able to immediately assess quality of the weld.

In a preferable manner the measures defined in claims 2 to 16 ensure that the welder's eyesight is protected against any injuries, wherein particularly a flash burn of the eyes is prevented.

The present invention will be explained in more detail by way of the enclosed schematic drawings.

Therein:

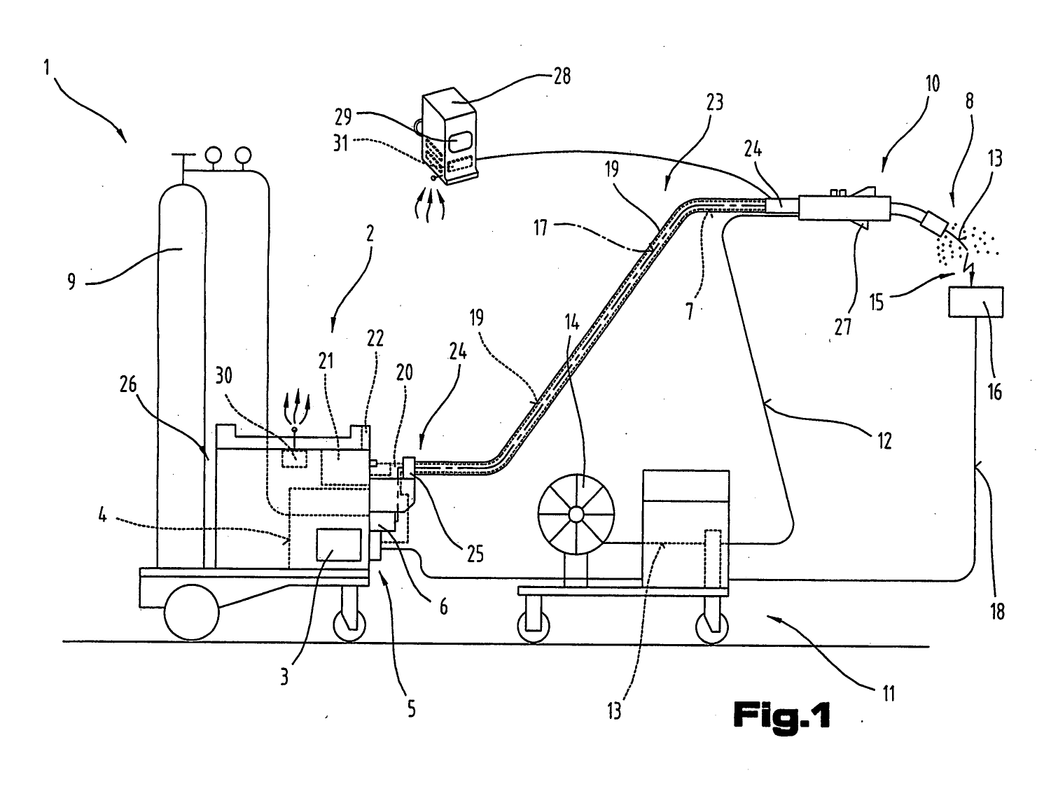

FIG. 1 shows a schematic representation of the welding plant with a welding device and a welding helmet connected thereto;

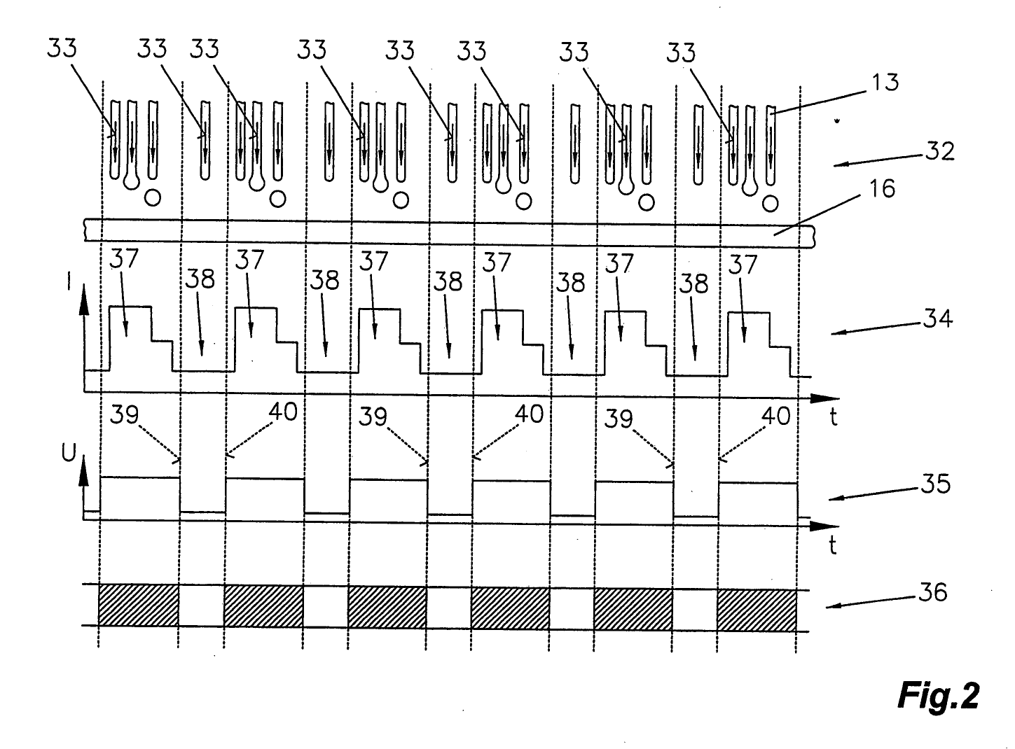

FIG. 2 shows a schematic representation of a pulse-welding process;

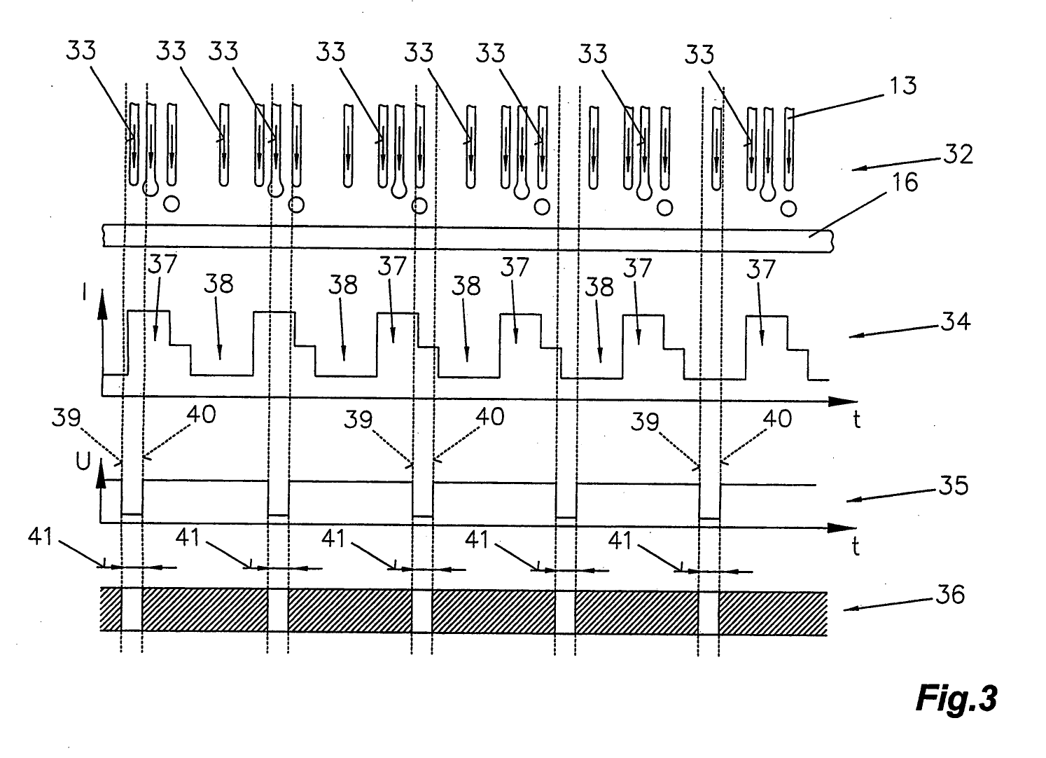

FIG. 3 shows a schematic representation of a further pulse-welding process with a different method of brightening;

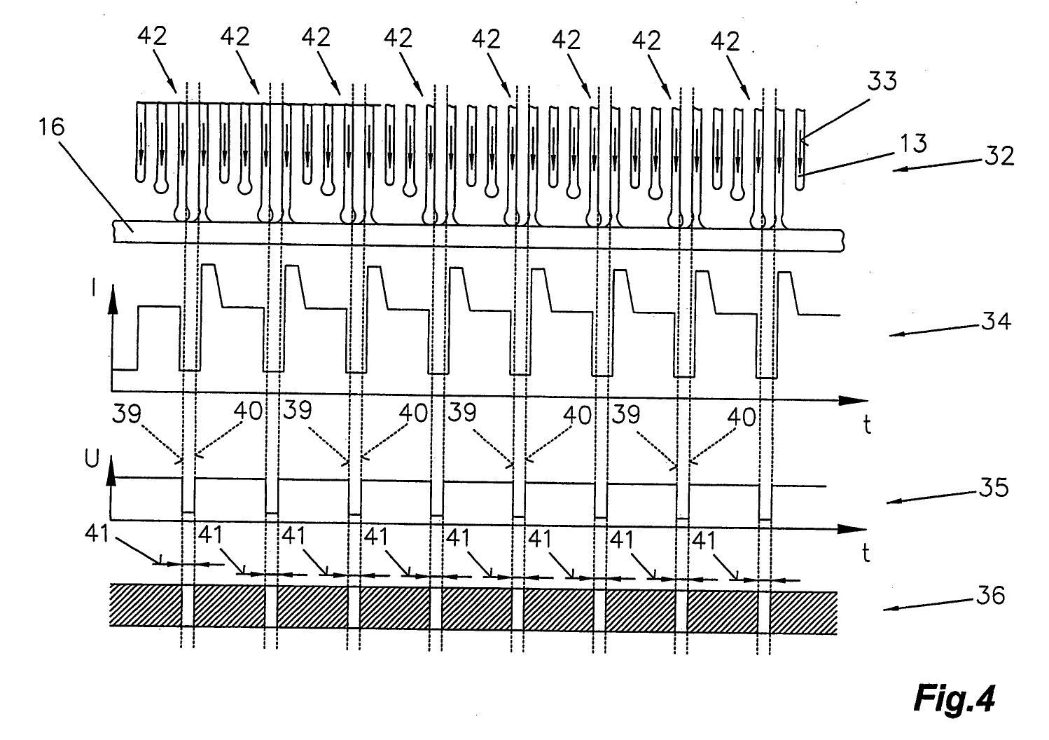

FIG. 4 shows a schematic representation of a short-circuit welding process;

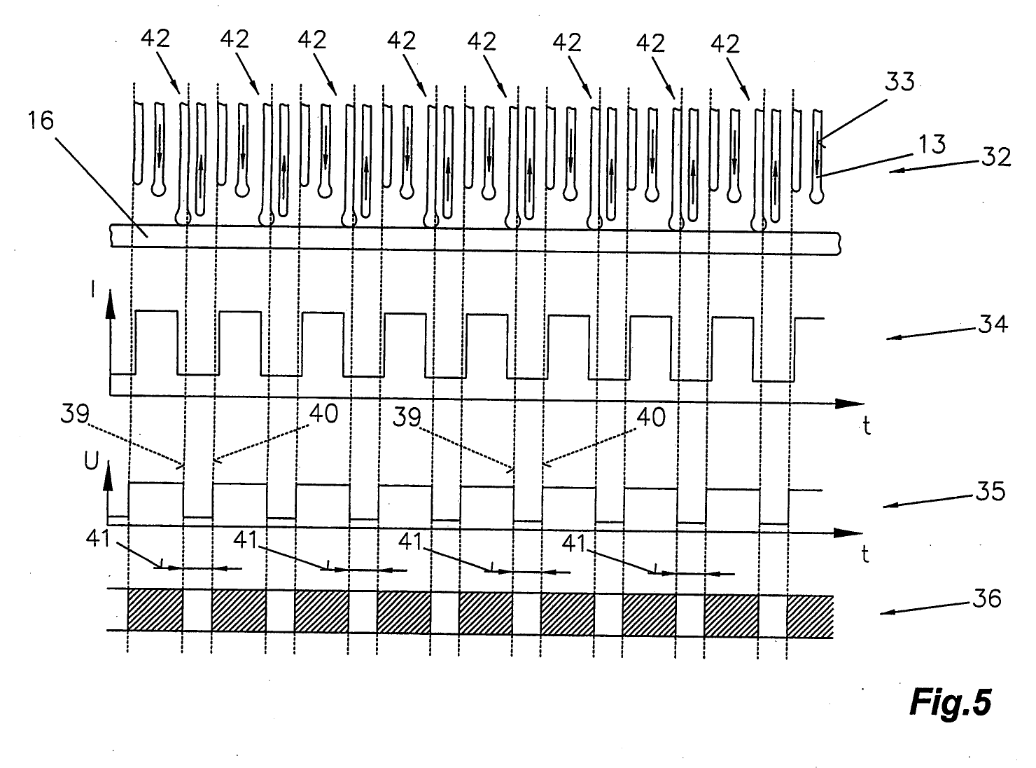

FIG. 5 shows a schematic representation of a CMT welding process;

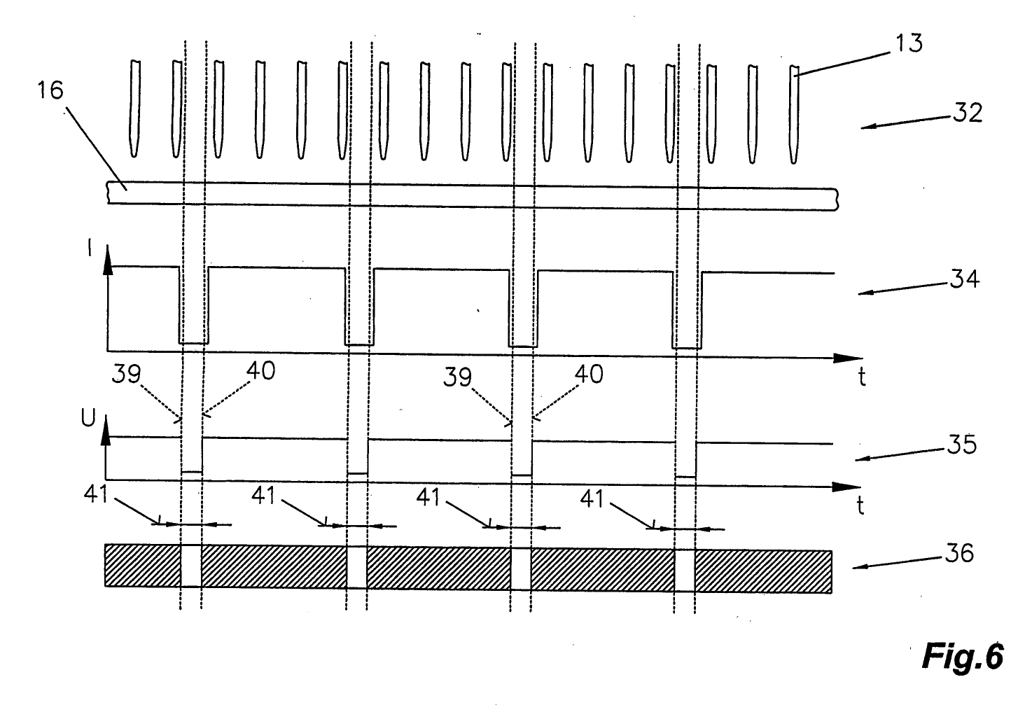

FIG. 6 shows a schematic representation of a WIG welding process; and

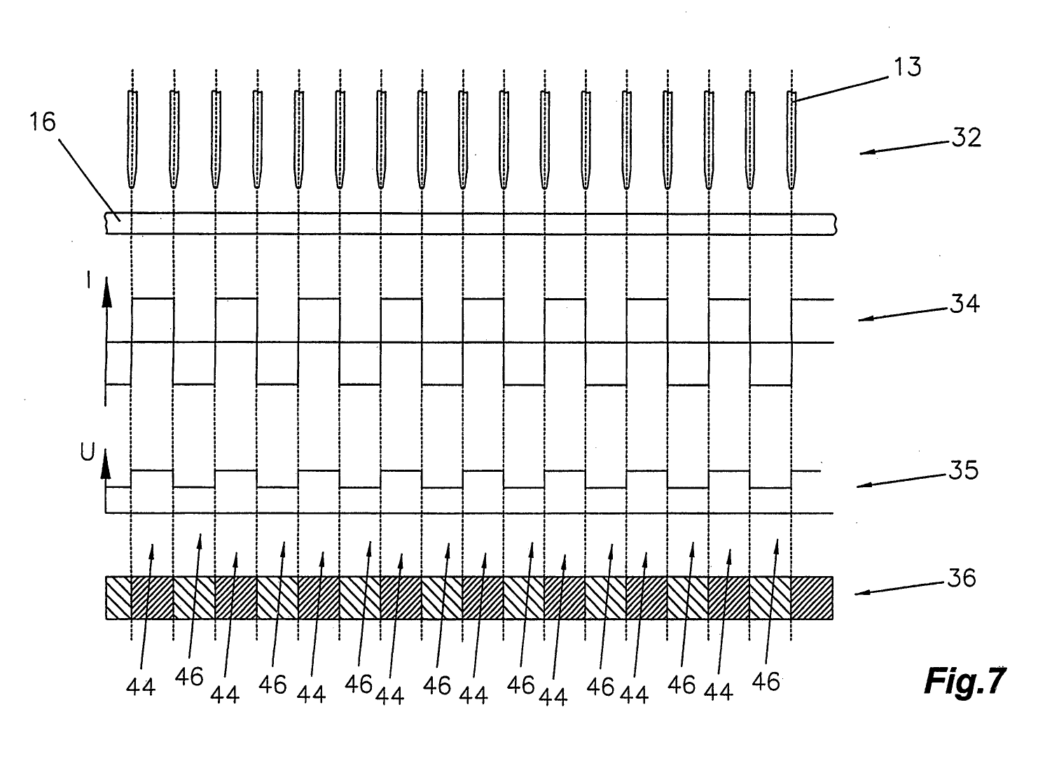

FIG. 7 shows a schematic representation of a AC-WIG welding process.

In FIG. 1 there is shown a welding plant or a welding device 1 for the most different welding processes, such as MIG/MAG welding and/or TIG welding or electrode-welding processes with or without protective-gas atmosphere. Certainly, it is possible to use the inventive solution with a current source and/or a welding current source or with robot welding plants.

The welding device 1 comprises a current source 2 with a power element 3, a control device 4 and a switching member 5 associated to the power element 3 and/or the control device 4. The switching member 5 and/or the control device 4 is/are connected with a control valve 6 which is arranged in a supply line 7 for a gas 8, in particular a protective gas, such as, e.g. CO₂, helium, argon or the like, between a gas reservoir 9 and a welding torch 10.

Moreover, also a wire feeder 11, which is common with, e.g. MIG/MAG welding, can be activated via the control device 4, wherein a welding wire 13 is supplied from a feed drum 14 to the area of the welding torch 10 via a supply line 12. Of course, it is possible to integrate the wire feeder 11 in the welding device 1, in particular in the basic housing, as known from the prior art, and not to design it as an accessory device, as shown in FIG. 1.

The current for establishing an electric arc 15 between the welding wire 13 and a workpiece 16 is supplied from the power element 3 to the welding torch 10 and/or the welding wire 13 via a welding line 17, wherein the workpiece 16 to be welded is also connected with the welding device 1, in particular the power element 3, via a further welding line 18 and, thus, an electric circuit can be established via the electric arc 15.

For cooling the welding torch 10, the welding torch 10 can be connected with a liquid reservoir via a cooling circiut 19, in particular a water reservoir 21, a flow monitor 20 being interposed, whereby when the welding torch 10 is put into operation, the cooling circuit 19, in particular a liquid pump used for the liquid present in the water reservoir 21, will be started and, thus, cooling of the welding torch 10 and/or the welding wire can be effected.

The welding device 1 further comprises an input and/or output device 22, via which the most different welding parameters and operation modes of the welding device 1 can be adjusted. In doing so, the welding parameters adjusted via the input and/or output device 22 are forwarded to the control device 4 and, subsequently, the latter will activate the individual components of the welding plant or welding device 1.

Furthermore, in the exemplary embodiment illustrated, the welding torch 10 is connected with the welding device 1 or the welding plant via a hose package 23. In the hose package 23, the individual lines extending from the welding device 1 to the welding torch 10 are arranged. The hose package 23 is connected with the welding torch 10 via a prior-art connecting device 24, whereas the individual lines in the hose package 23 are connected with the individual contacts of the welding device 1 via jacks and/or plug-in connections. In order to ensure an appropriate strain relief of the hose package 23 the latter is connected with a housing 26, preferably with the basic housing of the welding device 1, via a strain-relief device 25.

In order to allow for the welding process to be started by the user or welder, a starting switch 27 is provided on the welding torch 10, that is, a signal is generated by the welder by activating the starting switch 27, said signal being forwarded to the control device 4 via at least one line, so that the control device 4 can detect that a welding process and, thus, an ignition of the electric arc 15, is to be started such that all process steps necessary, such as starting a gas preflow, igniting the electric arc 15, activating the wire feeder 11 etc., can be introduced by the control device 4. These individual process steps are already known per se from the prior art and this is why they will be not described in more detail. Certainly, it is possible that the starting switch 27 is provided on the input and/or output device 22 or be provided additionally on the same, instead of being arranged on the welding torch 10.

In order to protect the welder, in particular his eyes, against the electric arc 15, in particular against the high light intensity of the electric arc 15, and/or against arising welding spatters, the welder uses an appropriate safety device, in particular a welding helmet 28 which, as already known from the prior art, is fastened on the head of the welder via a supporting structure, or which is held in front of one's face, in particular in front of one's eyes, by means of a handle. In the illustrated welding plant with the welding device 1 and the welding helmet 28, the latter comprises an electrically controlable protective shield 29, that is, darkening of the protective shield 29 is prompted by applying energy, in particular current and voltage, and, thus, the user can be protected against the light intensity of the electric arc 15.

As already known from the prior art, there is known a wired or wireless signal connection, in particular a data connection, between the welding helmet 28 and the welding device 1, wherein the protective shield 29 of the welding helmet 28 is darkened by the welding device 1 by transmitting a data signal. To this end, a transmitting/receiving apparatus 30 and/or 31 is/are arranged both in the welding device 1 and the welding helmet 28, wherein the transmitting/receiving apparatus 31 in the welding helmet 28 also activates the protective shield 29. In doing so, in the prior art, a starting signal is sent to the control device 4 of the welding device 1 by actuating the starting switch 27 on the welding torch 10, or on the welding device 1, for activation of a welding process, whereupon a data signal and/or a radio signal, or a starting command, will be generated by the control device 4 of the welding device 1 for the welding helmet 28. This data signal is then sent to the additional transmitting/receiving apparatus 31 integrated in the welding helmet 28 via the transmitting/receiving apparatus 30 integrated in the welding device 1, whereupon an activation device provided in the transmitting/receiving apparatus 31 will activate the electrically controlable protective shield 29, and darkening of the protective shield 29 will be introduced, whereupon, subsequently, the welding process, in particular ignition of the electric arc 15, will be started, preferably before expiry of a preadjustable preflow time and/or gas preflow time during which the protective shield 29 is darkened. Here, it is also possible that the electric arc 15 is ignited only after response of the transmitting/receiving apparatus 31, said response being realised by simple transmittance of a response signal via the transmitting and/or receiving apparatus 31, whereby it is ensured in any case that the protective shield 29 has already been darkened when the electric arc 15 is ignited.

According to the invention it is now provided that during the welding process, i.e. after ignition of the electric arc 15, the protective shield 29 is activated by a control device 4 of the welding device 1 via the transmitting/receiving apparatuses 30, 31 so that intensity and/or degree of darkening of the protective shield 29 is changed during the welding process, that is, e.g. in case of an existing electric arc 15, the protective shield 29 will be brightened for a short time. Thus, it is achieved that due to the different degrees of brightness, an optical brightening of the protective shield 29 is provided for the eye, without causing a flash burn of the eyesight. Thus, the user can much better observe the welding process and monitor specific welding states and, hence, achieve an optimum adjustment and an optimum welding result.

In order to avoid injury of the user's eyes, care must be taken with such a procedure that the control device 4 appropriately controls the welding process. Basically, uncontrolled states may occur during welding processes, such as a short circuit which is subsequently opened by high current, resulting in a very strong and intensive electric arc 15 after opening. Such strong and intensive electric arcs 15 must thus be prevented by the control device 4 to protect the welder's eye. For this purpose, the control device 4 appropriately activates the current source 2 of the welding device 1 such that at low intensity of darkening of the protective shield 29, i.e. when the screen of the protective shield 29 is opened, an increase of the current by the current source 2 of the welding device 1 will be prevented. It is further provided that prior to reducing or changing the intensity of darkening of the protective shield 29, the current level provided by the current source 2 is determined by the control device 4. Thus, the control device 4 may set the degree of darkening for the protective shield 29 with respect to the presently applied current, and the control device 4 may check whether a change in the degree of darkening of the adjusted degree of darkening may be effected at the presently applied and supplied current level without any danger for the user. If this is not the case, the control device 4 may discontinue or postpone the procedure of changing the degree or intensity of darkening of the protective shield 29 or may appropriately activate the current source 2 such that the current may be reduced to a value which corresponds to the adjusted degree of darkening or to the degree of darkening to be changed.

Consequently, it can basically be stated that the protective shield 29 is activated as a function of the process state of the welding process, that is, during the welding process the protective shield 29 is repetitively activated for changing the degree or intensity of darkening of the protective shield 29, wherein at certain preadjustable process states the protective shield 29 is activated and/or the welding process is appropriately controlled for changing the degree of darkening. Of course, it is also possible that the protective shield 29 is activated at fixedly predetermined points of time, wherein, to this end, no attention is paid to the present process state. It should only be mentioned that preferably the control device 4 does an appropriate monitoring so that no injuries of the user's eyesight can occur.

Preferably, when the protective shield 29 is activated during the welding process, intensity of darkening of the protective shield is changed depending on the power of the present welding process. In doing so, when the protective shield 29 is activated, adjustment of darkening is effected between two or more stages, wherein the degree of darkening is transmitted to the transmitting/receiving apparatus 31 of the welding helmet 28 by means of a corresponding data signal. Here, the data exchange between the welding device 1 and the welding helmet may be effected by analog or digital data signals.

In FIGS. 2 to 7, diagrams of a sequence for the inventive darkening of the protective shield 29 during the welding process are illustrated in a simplified manner. Therein, the movement 32 of the welding wire is shown with respect to the workpiece 16, wherein the supply direction is indicated corresponding to the arrows 33. Furthermore, a current path 34 is illustrated for the different welding processes known, wherein illustration of ignition of the electric arc 15 and, thus, the first darkening of the protective shield 29 has been omitted, that is, a partial section of the current path 34 during the welding process is shown. In the next line of the diagram the data signal 35 is illustrated which is sent by the welding device 1 to the welding helmet 28 for activation of the protective shield 29, wherein the degree 36 of brightness of the protective shield 29 is schematically illustrated in the following representation in FIGS. 2 to 7. Therein, a narrower hatching means a high degree of darkening and a wider or no hatching stands for a low degree of darkening.

In FIG. 2 a pulse-welding process is schematically illustrated, wherein no more further details are given on the exact current path 34 since it is already known from the prior art.

In case of a pulse-welding process the protective shield 29 is darkened stronger in a pulse phase 37 than in a basic phase 38, that is, the control device 4 which is informed about the present process state changes the data signal 35 at a certain fixed point of time 39, as illustrated by dashed line, after drop separation has been completed, whereby the degree or intensity of darkening of the protective shield 29 is correspondingly changed by the transmitting/receiving apparatus 31 provided in the welding helmet 28. However, in order to provide protection of the user's eyes prior to the next pulse phase 37, wherein the welding current is again increased and the electric-arc intensity in turn increases, the data signal 35 is once again changed at a defined point of time 40, whereupon a darkening of the protective shield 29 will be effected, whereupon the next pulse phase 37 may be introduced.

Consequently, the protective shield 29 is always repetitively brightened during the basic phase 38 since a lower current strength is applied in the basic phase 38 than in the pulse phase and the electric-arc intensity is thus lower.

In FIG. 3, again, a pulse-welding process is illustrated, with a brightening of the protective shield 29 being however effected at different process states and points of time.

Here, care must be taken that the parameters for brightening the protective shield 29 are chosen such that no injuries of the user's eyes may occur, that is, when brightening the protective shield 29 at a high current strength, it is brightened for a very short time only; cf. period 41 in FIG. 3. In this context, it is also possible that the period 41 is changed for brightening depending on the current level, i.e. a dependency of the current level on the period 41 is created for brightening.

As can be seen from FIG. 3, the protective shield is brightened in each case at different points of time of the process, and process states, during the whole recurring process cycle, that is, e.g. a first brightening is started shortly before indroduction of the pulse phase 37 (cf. first point of time 39 in FIG. 3) and is effected over a pre-defined period 41 such that brightening is stopped at the point of time 40 after the current level for the pulse phase 37 has been reached. Now, the next brightening of the protective shield 29 is again effected in the next following pulse phase 37 but in contrast to the previous pulse phase 37 at offset points of times 39, 40. Such a timely offset brightening of the protective shield 29 continuous for the whole welding process, whereby the protective shield 29 will be shortly opened in all different process states and the user can thus better observe the different process states. Certainly, it is also possible that such a timely offset brightening is effected always in the pulse phase 37 only and no brightening is effected in the basic phase 38.

By such a timely shifting or offsetting of the brightening at different points of time of the process, it is achieved that the user receives the impression as in the case of a high-speed video.

In FIG. 4 a further, prior-art welding process per se, namely a short-circuit process, is illustrated.

In such a short-circuit process, a short circuit between the welding wire 13 and the workpiece 16 is usually effected at irregular intervals (an optimum process sequence is illustrated). Here, the short circuit is usually opened in the prior art by increasing the welding current.

Here, for brightening the protective shield 29, it is however intervened in the common process control since the welding device 1 has been adjusted e.g. such that the protective shield 29 is brightened in each short circuit phase 42. Thus, the current source 2 is now activated by the control device 4 after detecting the short circuit such that the current is reduced after the short circuit has occurred, whereupon the data signal 35 will be changed for changing the degree or intensity of darkening of the protective shield 29 and the protective shield 29 will thus be brightened. The brightening is maintained over a preadjusted period 41, whereupon the protective shield 29 will be again darkened and again released by the control device 4 for controlling the welding process so that the short circuit may be opened by a corresponding increase in the current.

In FIG. 5, a novel welding process, in particular a CMT process, is illustrated, wherein the molten welding-wire material is transferred to the workpiece 16 in a short circuit, wherein, however, no increase in current is effected for the material separation but the molten material is separated from the welding wire 13 by reversing the wire-supply direction. Here, brightening of the protective shield 29 is again preferably effected in the short-circuit phase 42, whereby the welder may exactly observe separation of the material or may observe the same at low current after the short circuit has been opened.

In FIG. 6, a WIG process common per se is illustrated, wherein, to this end, it is again intervened in the usual process control for brightening the protective shield 29, that is, a brightening is effected at certain adjustable time intervals 41, wherein for this purpose the current source 2 is activated by the control device 4 such that the welding current is reduced, whereupon the data signal 35 will be changed and the protective shield 29 will be brightened for a defined period 41. Here, it is possible that brightening is effected periodically or coincidentally at the most different time intervals.

FIG. 7 shows an AC-WIG proccess, wherein, here, a different degree of darkening and/or a differently strong darkening of the protective shield 29 is effected for the positive and negative period 44, 46 since there are different electric-arc intensities during the different periods 44, 46. Thus, it is possible to change the degree of brightness of the protective shield 29 simultaneously with the periods, as schematically illustrated with different hatchings.

Consequently, it can basically be stated that brightening of the protective shield 29 can be effected based on different adjustments which can be used for all welding processes known and are not restricted to the previously described exemplary embodiments. Here, it is possible to correspondingly activate the protective shield 29 at a cyclically occurring process state at each period or that the control device 4 detects an occurrence of a short circuit in the welding process, whereupon intensity of darkening of the protective shield 29 will be reduced during or preferably shortly after the short circuit has been opened or until ignition of the electric arc 15. It is also possible that the control device 4 changes the intensity of darkening depending on whether a positive or negative potential is applied to an electrode and/or on the welding wire 13, wherein there is a lower darkening in case of a positive potential than in case of a negative potential. It is also possible for all welding processes known that the protective shield 29 is brightened at different points of time of a recurring process state for achieving the effect of a high-speed video.

By such a control of the protective shield 29 of a protective helmet 28 it is achieved that a brightness of the protective shield 29 which is optically effective for the user is adjusted/reached by duration of brightening and/or by the most different intensities of darkening, wherein the control device 4 introduces a corresponding process state, in particular a state of brightening, for brightening the protective shield 29, wherein, during introduction of the state of brightening, the control device 4 sends respective deposited and adjustable parameters to the current source 2 for controlling the welding process and to the welding helmet 28 for adjusting brightness of the protective shield 29.

Of course, it is also possible that a separate control device is arranged in the welding helmet 28, which control device is connected with the control device 4 of the welding device 1. Thus, even additional functions can be performed in the welding helmet 28, e.g. a visual display of welding parameters on the protective shield 29. It can also be used with a robot plant since a robot with a welding device 1 is mostly arranged in a robot cell, wherein the robot cell comprises a viewing window. If this viewing window is designed as is the protective shield 29, the viewing window will be able to be correspondingly activated by means of the welding device 1.

It is further also possible that a photo sensor is additionally provided in the protective helmet 28, wherein the photo sensor is designed to detect the actual brightness. Here, it is then possible to transfer corresponding data to welding device 1 by the welding helmet 28. For this purpose the photo sensor can be used for safety functions if it is provided, e.g. behind the protective shield 29 and can thus be used for control and optimisation of shutter speeds. The photo sensor may, e.g. also be used as a turn-off emergency element in case the brightness exceeds a certain value.

1. A method for controlling a protective shield (29), e.g. on a welding helmet (28), of a welding device (1), wherein a signal is transferred by the welding device (1) to a transmitting/receiving apparatus (30), whereupon the electrically activatable protective shield (29) is activated by a transmitting/receiving apparatus (31) associated to the protective shield (29), e.g. integrated in the welding helmet (28), and the protective shield (29) is darkened, wherein during the welding process, i.e. after ignition of the electric arc (15), the protective shield (29) is activated by a control device (4) of the welding device (1) via the transmitting/receiving apparatuses (30, 31), wherein intensity and/or degree of darkening of the protective shield (29) is changed during the welding process by repetitively darkening and brightening the protective shield (29) in an alternating manner.

2. The method according to claim 1, wherein at low intensity of darkening of the protective shield (29), i.e. when the screen of the protective shield (29) is opened, an increase in the current by the current source (2) of the welding device 1 is prevented by the control device (4).

3. The method according to claim 1, wherein prior to reducing or changing the intensity of darkening, the current level provided by the current source (2) is determined by the control device (4).

4. The method according to claim 1, wherein darkening of of the protective shield (29) is changed as a function of the process state of the welding process.

5. The method according to claim 1, wherein the protective shield (29) is activated at fixedly predetermined points of time (39, 40).

6. The method according to claim 1, wherein darkening of the protective shield (29) is changed as a function of the power of the present welding process.

7. The method according to claim 1, wherein darkening of the protective shield (29) is changed between two or more states of the intensity of darkening.

8. The method according to claim 1, wherein the control device (4) detects when a short circuit, i.e. a short-circuit phase (42), occurs in the welding process, and in that the protective shield (29) is brightened during or preferably shortly after opening of the short circuit, or until ignition of the electric arc (15).

9. The method according to claim 1, wherein the intensity of darkening of the protective shield (29) is changed in the darkened and brightened phases as a function of the positive or negative potential applied to an electrode, wherein darkening is stronger in case of the positive potential during a positive period (44) than darkening in case of the negative potential during a negative period (46).

10. The method according to claim 1, wherein the protective shield (29) is activated corresponding to a cyclically occurring process state of the welding process.

11. The method according to claim 1, wherein in case of a pulse-welding process, wherein a pulse phase (37) and a basic phase (38) are cyclically alternated, the protective shield (29) is brightened during the basic phase (38).

12. The method according to claim 1, wherein a brightness of the protective shield (29) which is optically effective for the user is adjusted by the duration of the brightened phases of the protective shield (29).

13. The method according to claim 1, wherein a brightness of the protective shield (29) which is optically effective for the user is adjusted by the intensity of darkening.

14. The method according to claim 1, wherein a corresponding process state, in particular a state of brightening, is introduced by the control device (4) for brightening the protective shield (29).

15. The method according to claim 1, wherein respective deposited and adjustable parameters are transferred to the current source (2) by the control device (4) during introduction of the state of brightening.

16. The method according to claim 1, wherein the protective shield (29) is brightened at different points of time of a recurring process state.
